(19) United States
(12) Patent Application Publication
(10) Pub. No.: US 2008/0193982 A1
(43) Pub. Date: Aug. 14, 2008

(54) Production of Enzymes

(75) Inventors: Mads Peter Torry Smith, Raleigh, NC (US); Guillermo Coward-Kelly, Raleigh, NC (US); Keith Charles McFarland, Davis, CA (US); Derek Scott Akerhielm, Davis, CA (US)

Correspondence Address: NOVOZYMES NORTH AMERICA, INC., 500 FIFTH AVENUE, SUITE 1600, NEW YORK, NY, 10110, US

(73) Assignees: Novozymes Biotech, Inc., Davis, CA (US); Novozymes North America, Inc., Franklinton, NC (US)

(21) Appl. No.: 11/911,411

(22) PCT Filed: May 18, 2006
(86) PCT No.: PCT/US2006/019206

§ 371 Date: Oct. 12, 2007

Related U.S. Application Data
(60) Provisional application No. 60/682,718, filed on May 19, 2005.

(51) Int. Cl. C12P 21/06

(52) U.S. Cl. 435/71.2; 435/71.1

(57) ABSTRACT

The present invention relates to a process of producing desired enzyme(s) in a host cell, comprising cultivating said host cell capable of producing the desired enzyme(s) under conditions conducive for production of the desired enzyme(s) using a substrate for the host cell comprising liquefied and/or saccharified starch-containing plant material.

### TECHNICAL FIELD

The present invention relates to a process of producing desired enzymes in a host cell.

### BACKGROUND ART

Microbial host cells are today used extensively for producing enzymes by fermentation. Enzymes, especially for industrial use, e.g., enzymes for converting starch-containing plant material into syrups and/or fermentation products, are needed in large amounts, but can only be sold at relatively low prices. This renders the enzyme production cost an important factor for being successful in the market place. The substrate cost may constitute up to 40-50% of the total enzyme production cost. Substrates are available from commercial suppliers or may be prepared according to published compositions (e.g., in catalogues of the American Type Culture Collection). The most commonly used carbon source substrates for production of enzymes are purified glucose and similar sugars. Purified glucose and similar sugars are expensive. Therefore, there is a need for providing easily available and cheap substrates that can replace expensive purified substrates for production of desired enzymes used for especially industrial applications.

### SUMMARY OF THE INVENTION

It is an object of the present invention to provide a process of producing desired enzyme(s) in a host cell using an easily available and cheap substrate as substitute for purified glucose or similar substrates used today.

According to the first aspect, the invention relates to a process of producing desired enzyme(s) in a host cell, comprising cultivating said host cell capable of producing the desired enzyme(s) under conditions conducive for production of the desired enzyme(s) using a substrate for the host cell comprising liquefied and/or saccharified starch-containing plant material.

The invention also relates to the use of liquefied and/or saccharified starch-containing plant material for producing desired enzyme(s) in a host cell.

### BRIEF DESCRIPTION OF THE DRAWING

FIG. 1 shows SDS-PAGE of APE-[35˜40].

FIG. 2 shows PCS hydrolysis vs. enzyme (relative protein in broth/g cellulose) loading. APE-35 to APE-37 are glucose feed controls, with cellulose in feed and cellulose in batch as noted.

FIG. 3 shows PCS hydrolysis vs. enzyme (relative volume broth/g cellulose) loading. Runs APE-35 to APE-37 are glucose feed controls, with cellulose in feed and cellulose in batch as noted.

### DETAILED DISCLOSURE OF THE INVENTION

It is the object of the present invention to provide a process of producing desired enzyme(s) in a host cell using an easily available and cheap substrate that can replace expensive substrates, including purified glucose, used today.

The inventors have surprisingly found that liquefied and/or saccharified starch-containing plant material, such as corn mash (i.e., liquefied corn), may advantageously replace purified glucose or other similar sugars as carbon substrate in enzyme production processes. One of the advantages of the invention is that enzymes used for converting starch-containing plant material into desired syrups or fermentation products may be produced using substrates already available at the production site. In other words, the need for buying expensive carbon source substrates, such as purified glucose, from external suppliers can be avoided or at least reduced significantly as substrate for enzyme production is available on site.

### Process of Producing Enzymes

Production of enzymes in host cells of fungal origin, such as filamentous fungi, or bacterial origin is well known in the art. The process of the invention may be a well known process, except that the substrate used is liquefied and/or saccharified starch-containing plant material.

A host cell capable of producing the desired enzyme(s) is grown under precise cultural conditions at a particular growth rate. When the host cell culture is introduced into the fermentation medium the inoculated culture will pass through a number of stages. Initially growth does not occur. This period is referred to as the lag phase and may be considered a period of adaptation. During the next phase referred to as the “exponential phase” the growth rate of the host cell gradually increases. After a period of maximum growth the rate ceases and the culture enters the stationary phase. After a further period of time the culture enters the death phase and the number of viable cells declines. Most enzymes are produced in the exponential phase. However, enzymes may also be produced in the stationary phase or just before sporulation.

In other words, according to the invention the host cell is cultivated in a suitable medium and under conditions allowing the enzyme(s) to be expressed and preferably secreted and optionally recovered. The cultivation takes place in a fermentation medium comprising (a) substrate(s). According to the present invention the carbon substrate is liquefied and/or saccharified starch-containing plant material. Enzyme production procedures are well known in the art. Enzymes may be extracellular or intracellular. In context of the present invention the contemplated enzyme(s) is(are) preferably and mostly extracellular enzyme(s) secreted into the fermentation medium by the host cell. The enzyme(s) may be recovered using methods well known methods in the art. For example, in case of extracellular enzyme(s) recovery from the fermentation medium may be done by conventional procedures including, but not limited to, centrifugation, filtration, extraction, spray-drying, evaporation, or precipitation. Procedures for recovery of intracellular enzymes are also well known in the art.

At least in context of the present invention the term “cultivation” or “fermentation” means any process of producing (a) desired enzyme(s) using a mass culture of one or more host cells. The present invention is useful for industrial scale enzyme production, e.g., having a culture medium of at least 50 liters, preferably at least 100 liters, more preferably at least 500 liters, even more preferably at least 1000 liters, in particular at least 5000 liters.

The process of the invention may be performed as a batch, a fed-batch, a repeated fed-batch or a continuous process.

The process of the invention may be carried out aerobically or anaerobically. Some desired enzymes are produced by submerged cultivation and some by surface cultivation. Submerged cultivation is the most common for desired enzymes produced according to the invention.

Thus, according to the first aspect, the invention relates to a process of producing desired enzyme(s) in a host cell, comprising cultivating said host cell capable of producing the desired enzyme(s) under conditions conducive for production of the desired enzyme(s) using a substrate for the host cell comprising liquefied and/or saccharified starch-containing plant material.

### Substrate

The substrate used in a process of the invention may be any liquefied and/or saccharified starch-containing plant material. Preferred are starch-containing material selected from the group consisting of: tubers, roots and whole grain; and any combinations thereof. In an embodiment the starch-containing material is obtained from cereals. The starch-containing material may, e.g., be selected from the groups consisting of corn (maize), wheat, barley, cassava, sorghum, rye, milo, switch grass and potato, or any combination thereof. In a preferred embodiment the liquefied and/or saccharified plant material is corn mash. In general corn mash comprises 10-50 wt. % TS, preferably 25-40 wt. % TS (Total Solids). About 70 wt. % of the TS corn mash is starch.

In an embodiment the liquefied and/or saccharified starch-containing plant material is, at least partly, introduced into the fermentation tank(s) prior to starting up the enzyme production process (i.e., substrate filled into the tank(s)) and is used as carbon source during the initial incubation period. Preferably the liquefied and/or saccharified starch-containing plant material is, at least partly, added as substrate feed during the time span of the enzyme production process in significantly the same way as purified glucose is usually added in well known prior art processes. In general the great majority of the substrate is added as substrate feed. The optimal dosing of substrate feed depends on the enzyme(s) produced, the host cell, and/or the chosen process conditions. For instance, when producing cellulase using a strain of Trichoderma, such as Trichoderma reesei, as host cell the carbon source substrate level is kept low, i.e., below 1 g carbon source substrate/L, such as 1 g glucose/L. A process of the invention may last for the same period of time as a corresponding process using, e.g., purified glucose. Trichoderma fermentations in general last for between 5-9 days.

In an embodiment the liquefied and/or saccharified starch-containing plant material is filtered (i.e. Filtered Corn Mash (FCM), e.g., as described in the “Preparation of FCM” section in the “Materials & Methods” section below. The crudely filtered substrate allows easy pumping of the substrate into the feed lines. However, it is to be understood that filtering the liquefied and/or saccharified starch-containing plant material is not mandatory. However, if the concentration of the liquefied and/or saccharified substrate, e.g., FCM, is too dilute e.g., less than 200 g substrate/L, it may not be suitable as substrate feed. Therefore, the substrate/substrate feed may according to the invention be concentrated and/or may be combined with (an)other substrate(s) in order to increase the substrate concentration to a suitable level. It is preferred that the substrate feed concentration is above 200 g substrate/L, preferably above 400 g substrate/L, preferably above 500 g substrate/L, more preferably around 600 g substrate/L, e.g. between 300 and 800 g substrate/L, preferably between 400 and 700 g substrate/L, such as around 600 g substrate/L.

The liquefied and/or saccharified starch-containing plant material may be the only substrate or carbon source substrate added during the time span of producing the desired enzyme(s), or may constitute a significant percentage thereof, such as at least 10 wt. %, preferably at least 30 wt. %, more preferably at least 50 wt. %, more preferably at least 70 wt. %, even more preferably at least 90 wt. %, or most preferably at least 95 wt. % of all substrates or carbon source substrates.

Other carbon sources or substrates, such as (purified) glucose or similar substrates, may constitute the remaining part of the substrate comprising the liquefied and/or saccharified substrate. Nitrogen sources, inducers and other growth stimulator may be added to improve the fermentation and enzyme production. Nitrogen sources include ammonia (NH₄Cl) and peptides. Protease may be used, e.g., to digest protein to produce free amino nitrogen (FAN). Such free amino acids may function as nutrients for the host cell, thereby enhancing the growth and enzyme production. Preferred fermentation stimulators for growth include vitamins and minerals. Examples of vitamins include multivitamins, biotin, pantothenate, nicotinic acid, meso-inositol, thiamine, pyridoxine, para-aminobenzoic acid, folic acid, riboflavin, and Vitamins A, B, C, D, and E. Examples of minerals include minerals and mineral salts that can supply nutrients comprising P, K, Mg, S, Ca, Fe, Zn, Mn, and Cu.

Whether an inducer is added, and which inducer is added, depends on the host cell and desired enzyme(s) to be produced. For instance, when producing cellulases, cellulose is often used as inducer.

According to the invention the liquefied and/or saccharified starch-containing plant material (i.e. substrate) may be added to the culture medium either prior to inoculation or after inoculation of the host cell culture in an amount corresponding to the amount of purified glucose or similar carbon source substrate normally used (i.e. replaced/substituted according to the invention). This means that the liquefied and/or saccharified plant material may be added in an amount that equals that of glucose normally used. In other words, the liquefied and/or saccharified starch-containing plant material to glucose ratio (i.e., kg liquefied and/or saccharified plant material per kg glucose or other similar carbon source substrate) is about from 2:1 to 3:1. However, as mentioned above the liquefied and/or saccharified plant material may also be concentrated or combined with other substrates in order to obtain a suitable concentration as mentioned above.

As mentioned above the liquefied and/or saccharified starch-containing plant material is used the same way purified glucose is normally used as substrate in well known enzyme production processes. The concentration of substrate during a process of the invention corresponds to the glucose level in well known processes in the art, but depends to some extent on the desired enzyme(s). A person skilled in the art can easily determine which amount of substrate to add during a process of the invention. Further guidance may be found in for instance Biochemical Engineering Fundamentals by James E Bailey and David F. Ollis, Second Edition, McGraw-Hill Book Company 1986.

According to the invention “liquefied starch-containing plant material” means plant material that has been subjected to hydrolysis by amylase, such as an alpha-amylase, or acid treatment for a suitable period of time. In a preferred embodiment the plant material has been reduced in particle size, e.g., by dry or wet milling, before hydrolysis to increase accessibility to the surface of the plant material.

The starch-containing plant material or liquefied plant material may also be saccharified. “Saccharified” means that the plant material, e.g., maltodextrin (such as liquefied starch-containing plant material) or uncooked starch-containing plant material is converted to low molecular sugars, e.g., DP₁₋₃, such as glucose and maltose (i.e., carbohydrate source) that can be metabolized by the host cell in question.

An advantage of the invention is that the liquefied and/or saccharified plant material may be available on the enzyme production site. The process of the invention is especially suitable for production of enzymes at locations where starch-containing material is already converted into a suitable substrate. In other words, the process of the invention is especially suitable at location where, e.g., a glucose- or starch-containing process stream is produced for use in syrup production or in a fermentation process. However, the liquefied and/or saccharified plant material may also be produced for the purpose of being used as substrate for enzyme production according to the invention. The process of the invention is especially advantageous in cases where the liquefied and/or saccharified plant material is an intermediate product in, e.g., starch-to-syrup or starch-to-fermentation product processes. This makes the substrate especially easily available and cheap for on-site production of enzymes.

### Desired Enzyme(s)

The enzyme(s) produced according to a process of the invention may be any enzyme(s). Preferred enzymes are hydrolases (class EC 3 according to Enzyme Nomenclature), including especially cellulases, hemicellulases, amylases, glucoamylases or other hydrolases, especially used for converting plant materials into syrups and fermentation substrates, e.g., converted by a yeast into ethanol.

The enzyme may be homologous or heterologous to the host cell.

The term “homologous enzyme” means an enzyme encoded by a gene that is derived from the host cell in which it is produced.

The term “heterologous enzyme” means an enzyme encoded by a gene which is foreign to the host cell in which it is produced.

In one embodiment the desired enzyme is a mono-component enzyme. In another embodiment the desired enzyme is an enzyme preparation or enzyme complex consisting of two of more enzymes derived from a wild-type host cell or a mutant thereof. An example of an enzyme complex is the well known cellulase complex comprising endoglucanase, exo-cellobiohydrolase and beta-glucosidase. An example of an enzyme preparation is the above mentioned cellulase complex where one or more enzyme encoding genes, e.g., endoglucanase gene(s), have been deleted from the wild-type host cell. A cellulase complex or preparation may be produced by a wild-type host cell or mutant thereof. In one embodiment the enzyme(s) is(are) produced recombinantly in a suitable recombinant host cell different from the donor cell from which the enzyme coding gene is derived. The desired enzyme(s) may be extracellular or intracellular. Extracellular enzymes are preferred. A desired enzyme may also be a variant of a wild-type enzyme.

### Cellulase and Hemicellulase

A cellulase and/or hemicellulase may be the desired enzyme produced according to the invention.

Contemplated hemicellulases include xylanases, arabinofuranosidases, acetyl xylan esterases, glucuronidases, endo-galactanase, mannases, endo- or exo-arabinases, and exo-galactanses.

Contemplated cellulases include those of bacterial or fungal origin. Chemically modified or protein engineered variants are included. Suitable cellulases include cellulases from the genera Bacillus, Pseudomonas, Humicola, Fusarium, Thielavia, Acremonium, and Trichoderma, e.g., fungal cellulases produced by Humicola insolens, Myceliophthora thermophila, Thielavia terrestris, Fusarium oxysporum, and Trichoderma reesei. In a preferred embodiment the desired enzyme is the cellulase complex which is homologously produced by Trichoderma reesei.

In another preferred embodiment the desired enzyme is a cellulase preparation produced heterologously in Trichoderma reesei, wherein one or more hydrolases foreign to Trichoderma reesei are produced.

In another embodiment the desired enzyme is the cellulase complex which is homologously produced by Humicola insolens.

### Amylase

An amylase may be the desired enzyme produced according to the invention. Contemplated amylases include alpha-amylases, beta-amylases and maltogenic amylases.

An alpha-amylase may be derived from the genus Bacillus, such as, derived from a strain of B. licheniformis, B. amyloliquefaciens, B. sultilis and B. stearothermophilus. Other alpha-amylases include alpha-amylase derived from the strain Bacillus sp. NCIB 12289, NCIB 12512, NCIB 12513 or DSM 9375, all of which are described in detail in WO 95/26397, or the alpha-amylase described by Tsukamoto et al., Biochemical and Biophysical Research Communications, 151 (1988), pp. 25-31.

Other alpha-amylases include alpha-amylases derived from a filamentous fungus, preferably a strain of Aspergillus, such as, Aspergillus oryzae and Aspergillus niger.

In a preferred embodiment, the desired enzyme is an alpha-amylase derived from Aspergillus oryzae such as the one having the amino acid sequence shown in SEQ ID NO: 10 in WO 96/23874 which is hereby incorporated by reference).

The desired enzyme may also be an alpha-amylase derived from A. niger, especially the one disclosed as “AMYA_ASPNG” in the Swiss-prot/TeEMBL database under the primary accession no. P56271.

The desired enzyme may also be a beta-amylase, such as any of plants and micro-organism beta-amylases disclosed in W. M. Fogarty and C. T. Kelly, Progress in Industrial Microbiology, vol. 15, pp. 112-115, 1979 (which is hereby incorporated by reference).

The desired enzyme may also be a maltogenic amylase. A “maltogenic amylase” (glucan 1,4-alpha-maltohydrolase, E.C. 3.2.1.133) is able to hydrolyze amylose and amylopectin to maltose in the alpha-configuration. A specifically contemplated maltogenic amylase is the one derived from Bacillus stearothermophilus strain NCIB 11837. Maltogenic alpha-amylases are described in U.S. Pat. Nos. 4,598,048, 4,604,355 and 6,162,628, which are hereby incorporated by reference.

### Glucoamylase

A glucoamylase may be the desired enzyme produced according to the invention. A glucoamylase may be derived from any suitable source, e.g., derived from a micro-organism or a plant. Preferred glucoamylases are of fungal or bacterial origin, e.g., selected from the group consisting of Aspergillus glucoamylases, in particular the A. niger G1 or G2 glucoamylases (Boel et al., 1984, EMBO J. 3:5, p. 1097-1102); the A. awamori glucoamylase (WO 84/02921), A. oryzae glucoamylase (Agric. Biol. Chem., 1991, 55:4, p. 941-949). Other glucoamylases include Athelia rolfsii (previously denoted Corticium rolfsii) glucoamylase (see U.S. Pat. No. 4,727,026 and (Nagasaka, Y. et al. (1998) Purification and properties of the raw-starch-degrading glucoamylases from Corticium rolfsii, Appl Microbiol Biotechnol 50:323-330), Talaromyces glucoamylases, in particular, derived from Talaromyces emersonii (WO 99/28448), Talaromyces leycettanus (U.S. Pat. No. Re. 32,153), Talaromyces duponti, Talaromyces thermophilus (U.S. Pat. No. 4,587,215). Bacterial glucoamylases contemplated include glucoamylases from the genus Clostridium, in particular C. thermoamylolyticum (EP 135,138), and C. thermohydrosulfuricum (WO 86/01831).

### Host Cell Capable of Producing Enzymes

The host cell may be of any genus. As mentioned above the desired enzyme may be homologous or heterologous to the host cell capable of producing the desired enzyme(s).

The term “recombinant host cell”, as used herein, means a host cell which harbours gene(s) encoding the desired enzyme(s) and is capable of expressing said gene(s) to produce desired enzyme(s). The desired enzyme(s) coding gene(s) may be transformed, transfected, transducted, or the like, into the recombinant host cell using techniques well known in the art.

When the desired enzyme(s) is(are) heterologous enzyme(s) the recombinant host cell capable of producing the desired enzyme(s) is(are) preferably of fungal or bacterial origin. The choice of recombinant host cell will to a large extent depend upon the gene(s) coding for the desired enzyme(s) and the source of said enzyme(s).

The term “wild-type host cell”, as used herein, refers to a host cell that natively harbours gene(s) coding for the desired enzyme(s) and is capable of expressing said gene(s). When the desired enzyme(s) is(are) homologous enzyme preparations or complexes the wild-type host cell or mutant thereof capable of producing the desired enzyme(s) may preferably be of fungal or bacterial origin.

A “mutant thereof” may be a wild-type host cell in which one or more genes have been deleted, e.g., in order to enrich the desired enzyme preparation in a certain component. A mutant wild-type host cell may also be a wild-type host cell transformed with one or more additional genes coding for additional enzymes in order to introduce one or more additional enzyme activities into the desired enzyme complex or preparation natively produced by the wild-type host cell. The additional enzyme may have the same activity (e.g. cellulase activity) but merely be another enzyme molecule, e.g. with different properties. The mutant wild-type host cell may also have additional homologous enzyme coding genes transformed, transfected, transducted, or the like, preferably integrated into the genome, in order to increase expression of that gene to produce more enzyme.

In a preferred embodiment the recombinant or wild-type host cell is of filamentous fungus origin. Examples of host cells include the ones selected from the group comprising a Acremonium, Aspergillus, Aureobasidium, Bjerkandera, Ceriporiopsis, Coprinus, Coriolus, Cryptococcus, Filobasidium, Fusarium, Humicola, Magnaporthe, Mucor, Myceliophthora, Neocallimastix, Neurospora, Paecilomyces, Penicillium, Phanerochaete, Phlebia, Piromyces, Pleurotus, Schizophyllum, Talaromyces, Thermoascus, Thielavia, Tolypocladium, Trametes, or Trichoderma cell.

In a more preferred embodiment the filamentous fungal host cell is selected from the group comprising a strain of Aspergillus awamori, Aspergillus fumigatus, Aspergillus foetidus, Aspergillus japonicus, Aspergillus nidulans, Aspergillus niger or Aspergillus oryzae. In another preferred embodiment the filamentous fungal host cell is a strain of Fusarium bactridioides, Fusarium cerealis, Fusarium crookwellense, Fusarium culmorum, Fusarium graminearum, Fusarium graminum, Fusarium heterosporum, Fusarium negundi, Fusarium oxysporum, Fusarium reticulatum, Fusarium roseum, Fusarium sambucinum, Fusarium sarcochroum, Fusarium sporotrichioides, Fusarium sulphureum, Fusarium torulosum, Fusarium trichothecioides, or Fusarium venenatum cell. In another preferred embodiment, the filamentous fungal host cell is selected from the group comprising a strain of Bjerkandera adusta, Ceriporiopsis aneirina, Ceriporiopsis aneirina, Ceriporiopsis caregiea, Ceriporiopsis gilvescens, Ceriporiopsis pannocinta, Ceriporiopsis rivulosa, Ceriporiopsis subrufa, or Ceriporiopsis subvermispora, Coprinus cinereus, Coriolus hirsutus, Humicola insolens, Humicola lanuginosa, Mucor miehei, Myceliophthora thermophila, Neurospora crassa, Penicillium purpurogenum, Phanerochaete chrysosporium, Phlebia radiata, Pleurotus eryngii, Thielavia terrestris, Trametes villosa, Trametes versicolor, Trichoderma harzianum, Trichoderma koningii, Trichoderma longibrachiatum, Trichoderma reesei, or Trichoderma viridel.

In another preferred embodiment the recombinant or wild-type host cell is of bacterial origin. Examples of host cells include the ones selected from the group comprising gram positive bacteria such as a strain of Bacillus, e.g., Bacillus alkalophilus, Bacillus amyloliquefaciens, Bacillus brevis, Bacillus circulans, Bacillus coagulans, Bacillus lautus, Bacillus lentus, Bacillus licheniformis, Bacillus megaterium, Bacillus stearothermophilus, Bacillus subtilis, or Bacillus thuringiensis; or a Streptomyces strain, e.g., Streptomyces lividans or Streptomyces murinus; or from a gram negative bacterium, e.g., E. coli or Pseudomonas sp.

### Use

In the second aspect the invention relates to the use of liquefied and/or saccharified starch-containing plant material for producing a desired enzyme in a host cell.

The invention described and claimed herein is not to be limited in scope by the specific embodiments herein disclosed, since these embodiments are intended as illustrations of several aspects of the invention. Any equivalent embodiments are intended to be within the scope of this invention. Indeed, various modifications of the invention in addition to those shown and described herein will become apparent to those skilled in the art from the foregoing description. Such modifications are also intended to fall within the scope of the appended claims. In the case of conflict, the present disclosure, including definitions will be controlling.

Various references are cited herein, the disclosures of which are incorporated by reference in their entireties.

### Materials & Methods

### Materials

Trichoderma reesei SMA135-04 is disclosed in example 8 of the US patent publication no. 2005/0233423.

TRACE METALS PREPARATION

| Components | g/L |
|---|---|
| FeCl₃•6H₂0 | 216 |
| ZnSO₄•7H₂O | 58 |
| MnSO₄•H₂O | 27 |
| CuSO₄•5H₂O | 10 |
| H₃BO₃ | 2.4 |
| Citric Acid | 336 |

SEED FLASK PREPARATION

| Components | g/L |
|---|---|
| Glucose | 20 |
| Corn Steep Solids | 10 |
| (NH₄)₂SO₄ | 1.45 |
| KH₂PO₄ | 2.08 |
| CaCl₂•2H₂O | 0.36 |
| MgSO₄•7H₂O | 0.42 |
| Trace Metals (mL) | 0.2 |
| Pluronic ® L61 | (1-2) drops per SF |
| pH | 5 |
| Autoclave Time | 30 |
| (min) | |

SEED FLASK INOCULATION

| | |
|---|---|
| Inoculant (cm² of PDA Plate) | 1 |
| Seed Flask Volume (mL) | 100 |
| Incubation Time (hrs) | 45 |
| Shaker Temp | 28 |
| Shaker RPM | 200 |
| Transfer Criteria | pH < 4.0 |
| Vessel Inoculation Volume (mL) | 50 |

POST STERILE ADDITIONS (Foam Control Agent)

| Components | Amount | Sterile Method |
|---|---|---|
| 1:5 Pluronic ® L61:H₂O | 5 mL as needed | Autoclave |

### EXAMPLE 1

### Utility of Liquefied and Saccharified Corn Mash for Production of Cellulase Enzymes

Roughly ground corn (maize) grain was heated in water and treated with alpha-amylase (TERMAMYL™SC from Novozymes) to liquefy the thick mixture which contained approximately 34% dry weight solids. This mixture was saccharified by the addition of glucoamylase (SPIRIZYME™ FUEL from Novozymes) and incubation at 65° C. for 48 hours. This mixture was centrifuged at approximately 4500×g for 10 minutes to remove insoluble solids, filtered through Miracloth (Calbiochem), and the resulting filtered corn mash supernatant (FCM) was tested for sugar content by HPLC analysis using an Aminex HPX87H column (BioRad) eluted with 5 mM sulfuric acid with detection by a refractive index detector (Agilent). Standard solutions of Glucose, Cellobiose, Xylose and Ethanol were used to calibrate the quantitation of sugars and ethanol detected. Typical concentration of glucose in the sugary corn mash supernatant syrup was approximately 300 g/liter.

Fermentations were performed in Applikon 2L glass jacketed vessels, which have a working volume of 1.8 L. Temperature was measured by electronic thermocouples and controlled using a circulating water bath. Dissolved oxygen and pH were both measured using sensor probes purchased from Broadley James Corporation. An ADI 1030 controller allowed proportional feedback control to adjust pH using acid and base feed pumps based on a pH set-point and deadband. ADI 1012 stirrer controllers were used to drive an Applikon P310 motor to agitate the broth at speeds ranging from 1100 to 1300 rpm. Rushton radial-flow impellers were utilized without baffles. The broth was aerated using a sterile air flow at a rate of about 1 vvm; the air entered via a sparger located at the bottom of the tank, beneath the impeller.

The fermentations were run using the Trichoderma reesei strain SMA-135-04. Glycerol freezer stocks have been prepared and were used as inoculum for the seed flasks. Seed flasks were grown as shown in the table below. Some inocula were reduced in volume as shown.

Trichoderma reesei strain SMA-135-04 fermentation lasted for approximately 165 hours, at which time the tank was harvested. A glucose feed with cellulose in the feed to induce cellulase production was used. Pluronic® L61 surfactant (BASF) was used to reduce foaming as necessary. Examples with glucose feeds (APE-35, -36, 37) are compared to corn mash filtrate feeds (APE-38, -39, -40).

FERMENTATION MEDIUM

| | APE-35 | APE-36 | APE-37 | APE-38 | APE-39 | APE-40 |
|---|---|---|---|---|---|---|
| Components | g | g | g | g | g | g |
| Corn Steep | 18.0 | 18.0 | 18.0 | 18.0 | 18.0 | 18.0 |
| Solids | | | | | | |
| Cellulose | 75.0 | 150.0 | 150.0 | 75.0 | 150.0 | 150.0 |

| Components | g/L | g |
|---|---|---|
| Glucose | 4 | 7.2 |
| CaCl₂•2H₂O | 2.64 | 4.8 |
| (NH₄)₂SO₄ | 3.8 | 6.8 |
| KH₂PO₄ | 2.8 | 5.0 |
| MgSO₄•7H₂O | 1.63 | 2.9 |
| Trace Metals (mL) | 0.75 | 1.4 |
| Pluronic ® L61 (mL) | 1.8 | 3.2 |
| Media Volume | 1.8 | 1.8 |
| Added to Tank (L) | | |

| | |
|---|---|
| pH | 4.3 |
| Autoclave Time (min) | 60 |

FEED COMPOSITION

| | APE-35 | APE-36 | APE-37 | APE-38 | APE-39 | APE-40 |
|---|---|---|---|---|---|---|
| Components | g | g | g | mL | mL | mL |
| Glucose | 454.5 | 454.5 | 454.5 | | | |
| FCM | | | | 1500.0 | 1500.0 | 1500.0 |
| H₂O | 1500 mL | | | | | |
| | tot vol | | | | | |

OPERATING CONDITIONS

| Starting Volume (L) | 1.8 | |
|---|---|---|
| Temperature (° C.) | 28 | |
| pH | 4.75 ± 0.1 | |
| Starting Agitation (rpm) | 1100 | |
| Air Flow (VVM) | 1 | |
| Minimum DO (%) | 25 | 25 |

| | | Stan- | APE- | | APE- | APE- | APE- | |
|---|---|---|---|---|---|---|---|---|
| | Hrs | dard | 35 | APE-36 | 37 | 38 | 39 | APE-40 |
| Feed | 0 | 0 | 0 | 0 | 0 | 0 | 0 | 0 |
| (g wet | | | | | | | | |
| feed/hr) | | | | | | | | |
| | 18 | 3.6 | 1X | 0.75X | 1X | 0.5X | 0.5X | 0.75X |
| | 33 | 7.2 | 1X | 0.75X | 1X | 0.5X | 0.5X | 0.75X |

| | | | |
|---|---|---|---|
| pH | Acid | 5N | 1X = standard feed rate in g glucose/hr |
| Control | | H₃PO₄ | (maintain DO > 25% and glucose < 1 g/L) |
| | Base | 28% | |
| | | NH₄OH | |

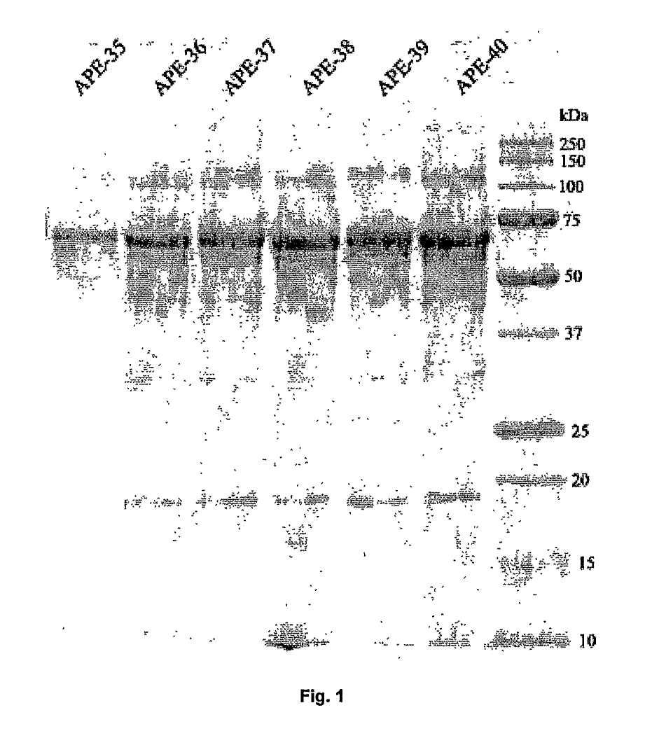

Aliquots of final fermentation broths were diluted 5 times in DDI water. Then 1 volume of diluted sample was mixed with 2 volumes of SDS sample buffer (BioRad) mixed with 5% beta-mercaptoethanol, boiled for 5 minutes. 15 MicroL of each sample was loaded onto 8-16% Tris-HCl gel (BioRad), electrophoresed and stained with BioSafe Coomassie Blue (FIG. 1). APE35-40 showed a clear band at ˜18 kD, some also showed a band at <10 kD. As seen on the gel, the protein expressed in APE-38, -39, and -40 were higher than the fermentations containing glucose.

The activities of enzyme broths were measured by their ability to hydrolyze dilute-acid pretreated corn stover (PCS) and produce sugars detectable by a chemical assay of their reducing ends. PCS was provided by the National Renewable Energy Laboratory (NREL, Golden Colo.) with glucan content of 53.2% (NREL data). 1 kg PCS was suspended in ˜20 liters of double deionized water in a bucket and, after the PCS settled, the water was decanted. This was repeated until the wash water is above pH 4.0, at which time the reducing sugars was lower than 0.06 g/L. The settled slurry was sieved through 100 Mesh screens to ensure ability to pipette. Percent dry weight content of the washed PCS was determined by drying the sample at a 105° C. oven for 24+ hours (until constant weight) and comparing to the wet weight.

PCS hydrolysis was performed in 96-deep-well plates (Axygen Scientific) sealed by a plate sealer (ALPS-300, ABgene). PCS concentration was 10 g/L, with 50 mM acetate pH 5.0. PCS hydrolysis was done at 50° C., with total reaction volume of 1.0 ml, without additional stirring. Each reaction was done in triplicates. Released reducing sugars were analyzed by p-hydroxy benzoic acid hydrazide (PHBAH) reagent as described below.

In detail, a 0.8 ml of PCS (12.5 g/L) was pipetted into each well of the 96-deep-well plates, to this 0.10 ml of sodium acetate buffer (0.5 M, pH 5.0) was added, then 0.10 ml diluted enzyme solution was added to start the reaction and to give the final reaction volume of 1.0 ml and PCS concentration of 10 g/L. The reaction mixture was mixed by inverting the deep-well plate at the beginning of hydrolysis and before taking each sample timepoint. After mixing, the deep-well plate was centrifuged (Sorvall RT7 with RTH-250 rotor) at 3000 rpm for 2 min before 20 microL of hydrolysate (supernatant) was removed and added to 180 microL of 0.4% NaOH in a 96-well microplate. This stopped solution was further diluted into the proper range of reducing sugars if necessary. The reducing sugars released were assayed by para-hydroxy benzoic acid hydrazide reagent (PHBAH, Sigma, 4-hydroxy benzyhydrazide): 50 microL PHBAH reagent (1.5%) was mixed with 100 microl sample in a V-bottom 96-well Thermowell plate (Costar 6511), incubated on a plate heating block at 95° C. for 10 min, then 50 microL DDI water was added to each well, mixed and 100 microL was transferred to another flat-bottom 96-well plate (Costar 9017) and absorbance read at 410 nm. Reducing sugar was calculated using a glucose calibration curve under the same conditions. Percent conversion of cellulose to reducing sugars was calculated as:

% conversion=reducing sugars(mg/ml)/(cellulose added(mg/ml)×1.11)

The factor 1.11 corrects for the weight gain in hydrolyzing cellulose to glucose.

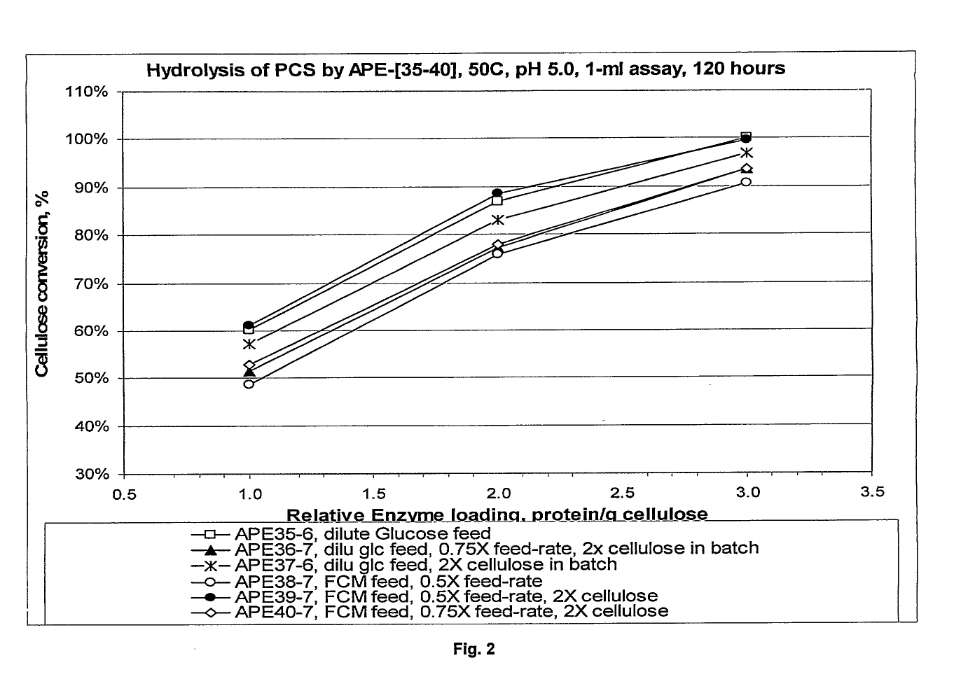

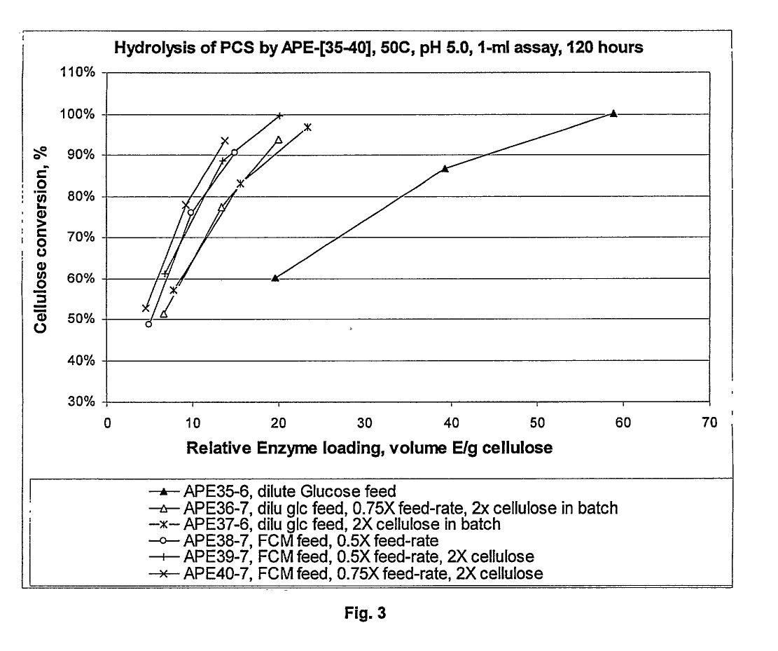

APE-39, producing the second highest protein levels (FIG. 3), also produced the most effective cellulase mixture of the fermentations utilizing corn mash (FIG. 2). All fermentations showed production of cellulase activity.

1-22. (canceled)

23. A process of producing an enzyme(s), comprising cultivating a host cell capable of producing the enzyme(s) under conditions conducive for production of the enzyme(s) using a substrate for the host cell comprising liquefied and/or saccharified starch-containing plant material.

24. The process of claim 23, wherein the host cell is of bacterial or fungal origin.

25. The process of claim 23, wherein the enzyme(s) is a hydrolase (class EC 3 according to Enzyme Nomenclature).

26. The process of claim 23, wherein the enzyme(s) is selected from the group consisting of amylase, cellulase, glucoamylase, and hemicellulase.

27. The process of claim 23, wherein the enzyme(s) is a cellulase complex/preparation.

28. The process of claim 27, wherein the cellulase complex/preparation is obtained from Trichoderma reesei.

29. The process of claim 27, wherein the cellulase complex/preparation is obtained from Humicola insolens.

30. The process of claim 23, wherein the enzyme(s) is produced in a tank of at least 50 liters.

31. The process of claim 23, wherein the liquefied and/or saccharified starch-containing plant material constitutes at least 50% (w/w) of the substrate added over the time span of the cultivation.

32. The process of claim 23, wherein the liquefied and/or saccharified starch-containing plant material is filtered before use.

33. The process of claim 23, wherein the substrate is at least partly added as a substrate feed during the time span of the production process of the desired enzyme.

34. The process of claim 23, wherein the liquefied and/or saccharified starch-containing plant material is concentrated to a concentration above 200 g substrate/L before use.

35. The process of claim 23, wherein the substrate feed concentration is above 200 g substrate/L.

36. The process of claim 23, wherein the substrate concentration during cultivation is kept below 1 g substrate/L.

37. The process of claim 23, wherein the liquefied and/or saccharified starch-containing plant material is derived from tubers, roots or whole grain; or any combinations thereof.

38. The process of claim 37, wherein the starch-containing material is derived from corn, cassava, wheat, barley, rye, milo, switch grass, and potatoes; or any combination thereof.

39. The process of claim 23, wherein the liquefied and/or saccharified starch-containing plant material is a side or shared stream from a fermentation product production.

40. The process of claim 23, wherein the substrate is prepared from starch-containing plant material that has been liquefied using an alpha-amylase or subjected to acid treatment.

41. The process of claim 23, wherein the substrate is prepared from starch-containing plant material or liquefied starch-containing plant material that has been saccharified using a glucoamylase.

42. The process of claim 23, further comprising recovering the enzyme(s).
